(19) United States
(12) Patent Application Publication
(10) Pub. No.: US 2026/0334876 A1
(43) Pub. Date: Oct. 29, 2026

(54) BIOSIGNAL MEASUREMENT DEVICE

(71) Applicant: Meditrix Co., Ltd., Seoul (KR)

(72) Inventors: Dong Jun KIM, Seoul (KR); Sung woo KIM, Seoul (KR); Kwang Moo KIM, Seongnam-si, Gyeonggi-do (KR); Seung Kyun HONG, Gwangmyeong-si (KR); Chi Hoon HAN, Gwangmyeong-si (KR)

(73) Assignee: Meditrix Co., Ltd., Seoul (KR)

(21) Appl. No.: 19/634,206

(22) Filed: Mar. 31, 2026

(30) Foreign Application Priority Data
Apr. 29, 2025 (KR) 10-2025-0056143

(51) Int. Cl. A61B 5/00; A61B 5/256; A61B 5/291

(52) U.S. Cl. CPC A61B 5/6803; A61B 5/256; A61B 5/291; A61B 5/6814; A61B 2562/04; A61B 2562/14; A61B 2562/164

(57) ABSTRACT

The present invention provides a biosignal measurement device including a body unit and a measurement unit disposed on one side of the body unit and configured to contact a user's body to detect biosignals. The body unit includes a housing provided with a communication module and a controller, and a pair of elastic pressing members, each having one side connected to the housing and another side connected to the measurement unit, the pair of elastic pressing members configured to press the measurement unit toward the user's body.

### RELATED APPLICATION

This application claims the benefit of priority of Republic of Korea Patent Application No. 10-2025-0056143 filed on April 29, 2025, the contents of which are all incorporated by reference herein in their entirety.

### FIELD AND BACKGROUND OF THE INVENTION

The present invention relates to a biosignal measurement device, and more particularly, to a biosignal measurement device capable of bringing a plurality of electrodes for detecting biosignals into close contact with a user's body.

A biosignal measurement device is a device equipped with EEG (electroencephalography) and EMG (electromyography) sensors to measure biosignals of a user, and performs a function of detecting electrical signals through electrodes attached to a head or a specific body part and analyzing the detected signals.

EEG sensors are used to measure brain activity and identify the state of the nervous system, and EMG sensors are used to detect electrical activity of muscles and analyze muscle movements or abnormalities in the nervous system.

Recently, as shown in FIG. 1, biosignal measurement devices (1) combined with an HMD (Head-Mounted Display) have been actively researched and developed. Such devices are utilized in various application fields, including enhancing user interfaces in conjunction with VR (virtual reality) and AR (augmented reality) systems, and being used for neurofeedback and artificial intelligence-based physiological response analysis.

However, conventional biosignal measurement devices have a problem in that the contact force of electrodes varies depending on the shape of the user's head and body structure. In order to increase the accuracy of biosignal measurement, the electrodes must be in stable contact with the skin, but the contact force may not be uniform depending on the size or shape of the user's head, which may result in degraded signal measurement accuracy.

In particular, when the contact force is insufficient, noise increases, causing a problem in that it is difficult to derive reliable biosignal data.

Therefore, the present invention aims to develop a biosignal measurement device that can effectively accommodate various body structures of users.

### SUMMARY OF THE INVENTION

### Technical Problem

An object of the present invention is to provide a biosignal measurement device capable of stably bringing electrodes into contact with a user's body regardless of the shape of the user's head.

The objects of the present invention are not limited thereto, and other objects not mentioned herein will be clearly understood by those skilled in the art from the following description.

### Technical Solution

A biosignal measurement device according to an embodiment of the present invention includes a body unit and a measurement unit disposed on one side of the body unit and configured to contact a user's body to detect biosignals, wherein the body unit includes a housing provided with a communication module and a controller, and a pair of elastic pressing members, each having one side connected to the housing and another side connected to the measurement unit, the pair of elastic pressing members configured to press the measurement unit toward the user's body.

In addition, the elastic pressing member may include a rotation shaft fixed to the housing, a rotation bracket rotatably connected to the rotation shaft, and an elastic body configured to press the rotation bracket toward the user's body.

In addition, the measurement unit may include a flexible body having a curvature that is deformable along a contour of the user's body, and a plurality of electrodes disposed on a surface of the flexible body that contacts the user's body.

In addition, the plurality of electrodes may include a pair of first electrodes fixed to the housing, and a pair of second electrodes each fixed to a respective one of the pair of elastic pressing members.

In addition, the flexible body may be formed such that a region in which the first electrodes are disposed and a region in which the second electrodes are disposed have different inclination angles.

In addition, the first electrodes may be disposed so as to have an inclination angle different from that of the second electrodes.

In addition, the second electrodes may be configured such that a tilting angle thereof is changed by an elastic force of the elastic pressing members when worn by the user.

In addition, the body unit may have a coupling portion formed on one surface thereof, the coupling portion configured to engage with an HMD device.

### Advantageous Effects of Invention

According to an embodiment of the present invention, the biosignal measurement device has an effect of pressing the plurality of electrodes toward the user's body so that all of the electrodes can be maintained in close contact with the user's body.

The effects of the present invention are not limited to the effects described above, and effects not mentioned herein will be clearly understood by those skilled in the art from the present specification and the accompanying drawings.

### BRIEF DESCRIPTION OF THE SEVERAL VIEWS OF THE DRAWINGS

FIG. 1 is a view illustrating a state in which a conventional biosignal measurement device is combined with an HMD.

FIG. 2 is a perspective view of a biosignal measurement device according to an embodiment of the present invention.

FIG. 3 is an exploded perspective view of the biosignal measurement device according to an embodiment of the present invention.

FIG. 4 is an exploded plan view illustrating a part of a body unit and a measurement unit included in FIG. 3.

FIG. 5 is an exploded perspective view of an elastic pressing member of FIG. 3.

FIG. 6 is a perspective view of an elastic pressing member and a second electrode body according to an embodiment of the present invention.

FIG. 7 is a cross-sectional view taken along line A-A of FIG. 4.

FIG. 8 is a cross-sectional view taken along line A-A of FIG. 4, illustrating a state in which the device is worn by a user.

FIG. 9 is an exploded plan view of the measurement unit of FIG. 4.

FIG. 10 is a rear view of the biosignal measurement device according to an embodiment of the present invention.

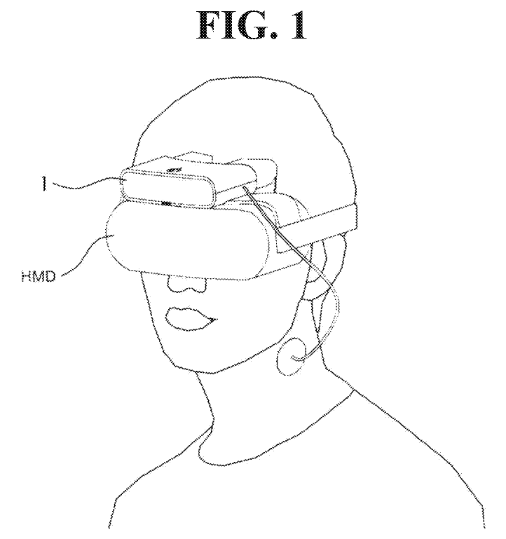

### DESCRIPTION OF SPECIFIC EMBODIMENTS OF THE INVENTION

The embodiments described in this specification and the configurations illustrated in the drawings are merely preferred examples of the disclosed invention, and various modifications may be made at the time of filing of the present application to replace the embodiments and drawings of this specification.

The same reference numerals or symbols used in the respective drawings of this specification denote parts or components that perform substantially the same functions. The shapes and sizes of elements in the drawings may be exaggerated for clarity of description.

The terminology used herein is for the purpose of describing embodiments and is not intended to limit and/or restrict the disclosed invention. Singular expressions include plural expressions unless the context clearly dictates otherwise. In this specification, terms such as "include" or "have" are intended to designate the presence of features, numbers, steps, operations, components, parts, or combinations thereof described in the specification, and do not preclude the presence or addition of one or more other features, numbers, steps, operations, components, parts, or combinations thereof.

Terms including ordinal numbers such as "first" and "second" used herein may be used to describe various components, but the components are not limited by such terms. The terms are used only to distinguish one component from another. For example, a first component may be named a second component, and similarly, a second component may also be named a first component without departing from the scope of the present invention.

Hereinafter, a biosignal measurement device 10 according to an embodiment of the present invention will be described in detail.

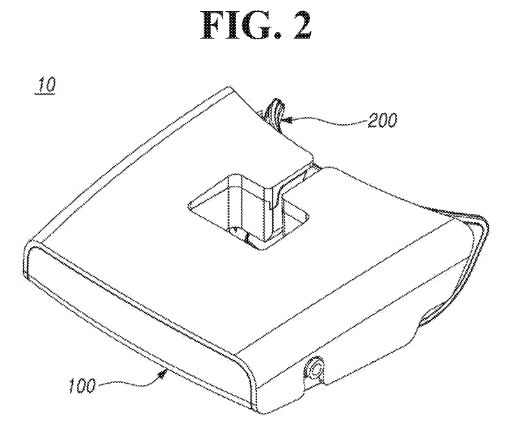

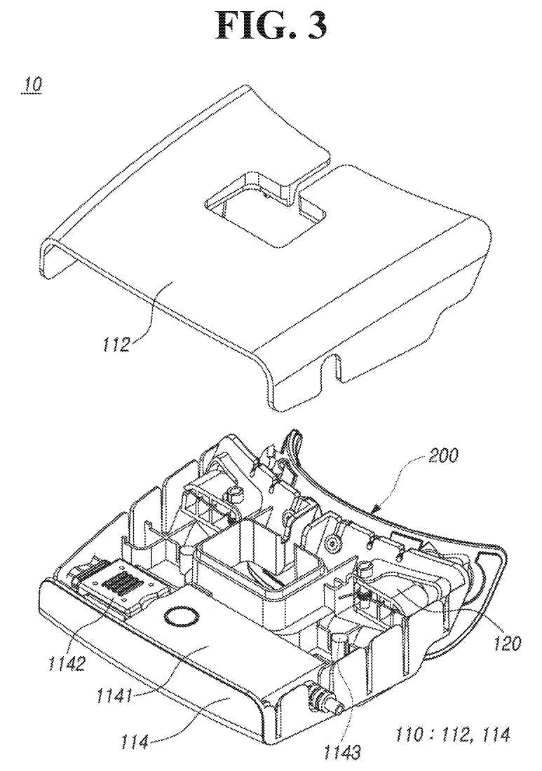

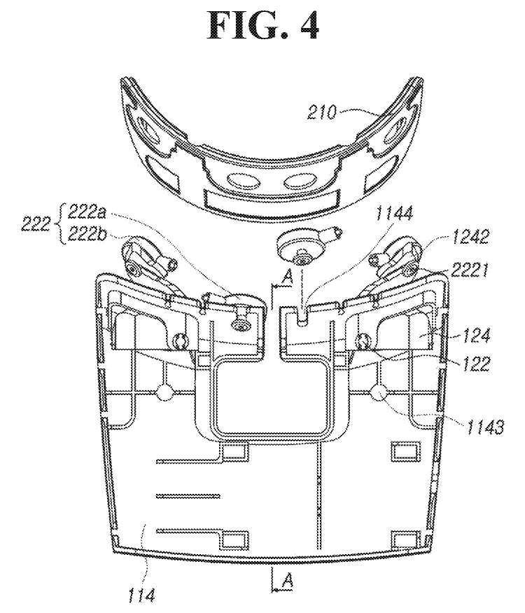

FIG. 2 is a perspective view of the biosignal measurement device according to an embodiment of the present invention. FIG. 3 is an exploded perspective view of the biosignal measurement device according to an embodiment of the present invention. FIG. 4 is an exploded plan view illustrating a part of a body unit and a measurement unit included in FIG. 3.

Referring to FIGS. 2 to 4, the biosignal measurement device 10 measures biosignals of a user (such as electrocardiogram, electromyogram, electroencephalogram, and blood pressure) and provides necessary information through another electronic device. The biosignal measurement device 10 includes a body unit 100 and a measurement unit 200.

The body unit 100 is capable of performing power supply, data storage, and communication. The body unit 100 includes a housing 110 and elastic pressing members 120.

The housing 110 may be provided with components necessary for operation of the biosignal measurement device 10. The housing 110 may be formed by coupling an upper cover 112 and a lower cover 114. The housing 110 may have a configuration coupled to an HMD (Head-Mounted Display) disposed on one surface thereof.

The lower cover 114 may be provided with a power supply unit 1141, a PCB 1142, a communication module (not shown), and the like therein. The PCB 1142 serves as a controller that processes the biosignals received from the measurement unit 200 and controls overall operations of the biosignal measurement device 10. A coupling portion 1143 configured to engage with an HMD (Head-Mounted Display) may be formed on one surface of the lower cover 114. For example, the coupling portion 1143 may be formed on a lower side of the lower cover 114 and fixed to an upper side of the HMD. The coupling portion 1143 may be formed as a groove into which a bolt is fastened. In addition, the lower cover 114 may have an insertion groove 1144 into which a first electrode body 222a is coupled.

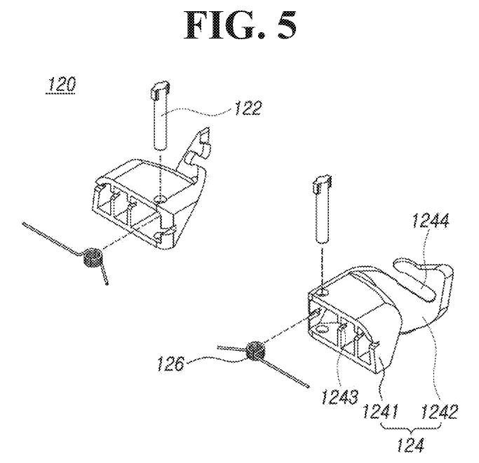

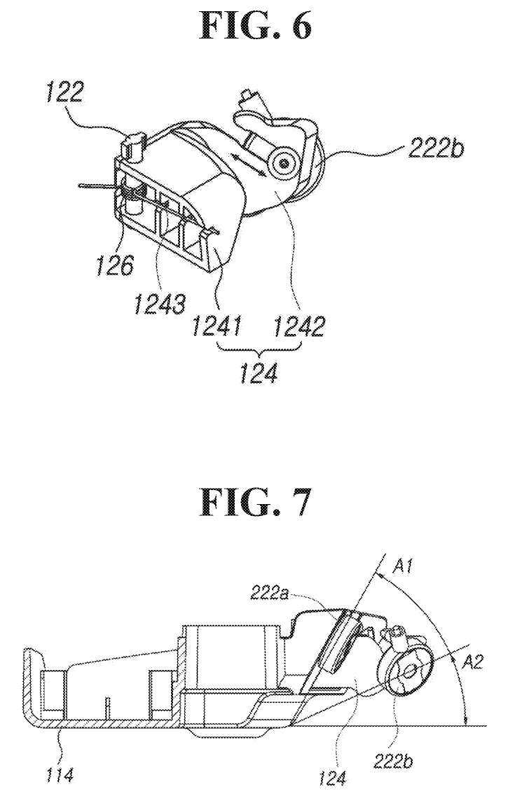

The elastic pressing members 120 each have one side connected to the housing 110 and another side connected to the measurement unit 200. The elastic pressing members 120 press the measurement unit 200 toward the user's body. FIG. 5 is an exploded perspective view of the elastic pressing member of FIG. 3. FIG. 6 is a perspective view of the elastic pressing member and a second electrode body according to an embodiment of the present invention.

Referring to FIGS. 5 and 6, the elastic pressing member 120 includes a rotation shaft 122, a rotation bracket 124, and an elastic body 126.

The rotation shaft 122 connects the lower cover 114 and the rotation bracket 124. The rotation shaft 122 is fixed to the lower cover 114. The rotation shafts 122 may be provided as a pair and disposed at symmetrical positions on the lower cover 114.

The rotation bracket 124 is rotatably connected to the rotation shaft 122. The rotation brackets 124 may be provided as a pair and disposed adjacent to both ends of the measurement unit 200.

The rotation bracket 124 may be coupled to the measurement unit 200. The rotation bracket 124 includes a bracket body 1241 and a support tab 1242.

The bracket body 1241 is rotatably connected to the rotation shaft 122. The bracket body 1241 is rotatable in a direction horizontal to the ground. The bracket body 1241 has an elastic body fixing groove 1243 into which the elastic body 126 is inserted.

The elastic body fixing groove 1243 is a groove in which the elastic body 126 is supported, and prevents the elastic body 126 in contact with the bracket body 1241 from being detached. The elastic body fixing groove 1243 may be disposed at an eccentric position of the bracket body 1241. For example, the elastic body fixing groove 1243 may be formed closer to an upper side than a lower side of the bracket body 1241, as shown in FIG. 6.

The support tab 1242 is fixed to the bracket body 1241. The support tab 1242 may be integrally formed with the bracket body 1241. A second electrode body 222b is coupled to the support tab 1242. The support tab 1242 has a support tab groove 1244 into which the second electrode body 222b is coupled. Here, the insertion groove 1144 and the support tab groove 1244 are formed to a predetermined depth so that the first electrode body 222a and the second electrode body 222b can move along a longitudinal direction thereof. In addition, the insertion groove 1144 and the support tab groove 1244 may be formed in different directions. For example, both the insertion groove 1144 and the support tab groove 1244 may be formed in a downward direction, but the support tab groove 1244 may be formed to face a downwardly inclined direction.

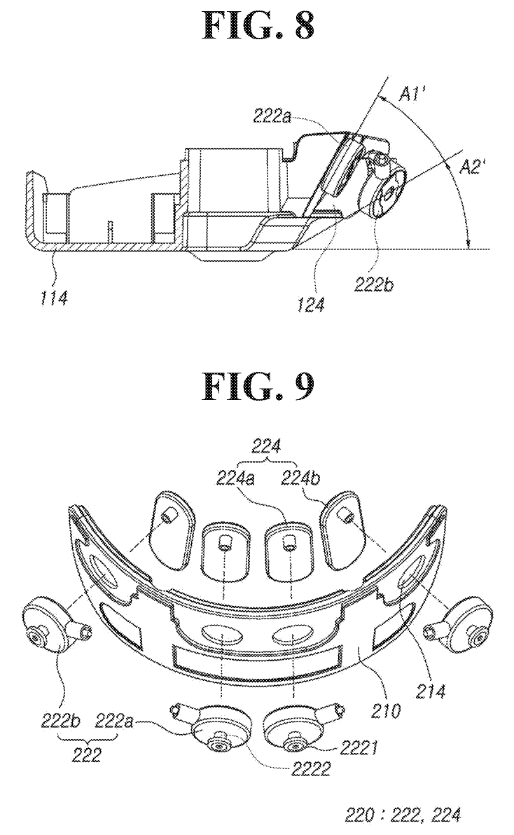

FIG. 7 is a cross-sectional view taken along line A-A of FIG. 4, and FIG. 8 is a cross-sectional view taken along line A-A of FIG. 4, illustrating a state in which the device is worn by a user.

Referring to FIGS. 7 and 8, an inclination angle A1 of the first electrode body 222a connected to the insertion groove 1144 may be formed to be greater than an inclination angle A2 of the second electrode body 222b connected to the support tab groove 1244. Accordingly, an inclination angle of a region of the flexible body 210 in contact with the first electrode body 222a is formed to be greater than an inclination angle of a region in contact with the second electrode body 222b, and a first electrode 224a connected to the first electrode body 222a may be formed to have an inclination angle greater than that of a second electrode 224b connected to the second electrode body 222b.

In addition, when worn by the user, the inclination angle of the first electrode body 222a may be maintained the same, while the inclination angle of the second electrode body 222b may be changed.

For example, the inclination angle A1 of the first electrode body 222a before the user wears the biosignal measurement device 10 may be the same as the inclination angle A1' of the first electrode body 222a after the user wears the biosignal measurement device 10.

On the other hand, the inclination angle A2 of the second electrode body 222b before the user wears the biosignal measurement device 10 may be smaller than the inclination angle A2' of the second electrode body 222b after the user wears the biosignal measurement device 10.

The elastic body 126 presses one side of the rotation bracket 124. A direction in which the elastic body 126 presses the rotation bracket 124 may be toward the user's body. For example, the elastic body 126 may be provided as a coil spring wound around the rotation shaft 122 to press the rotation bracket 124 toward the user's body. However, the elastic body 126 is not limited to a coil spring and may be provided in various forms capable of pressing the rotation bracket 124 in one direction. In addition, the elastic force with which the elastic body 126 presses the rotation bracket 124 is provided as a minimum force sufficient to bend the flexible body 210, and it will be understood that the elastic force is of a degree that allows the body to be pushed in a direction opposite to the direction in which the body contacts the plurality of electrodes 224 when the plurality of electrodes 224 are in contact with the user's forehead.

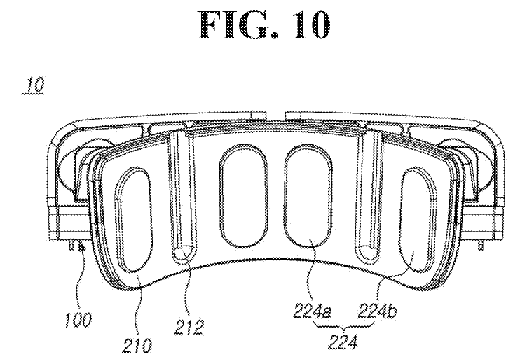

The measurement unit 200 is disposed on one side of the housing 110. The measurement unit 200 contacts the user's body to detect biosignals. The measurement unit 200 transmits the measured biosignals to the PCB 1142. Hereinafter, it will be described that a body part of the user to which the measurement unit 200 according to an embodiment of the present invention contacts is the forehead. FIG. 9 is an exploded plan view of the measurement unit of FIG. 4, and FIG. 10 is a rear view of the biosignal measurement device according to an embodiment of the present invention. Referring to FIGS. 9 and 10, the measurement unit 200 includes a flexible body 210 and an electrode unit 220.

The flexible body 210 contacts the user's forehead. The flexible body 210 may be formed of a soft material having elasticity. For example, the flexible body 210 may be formed of a silicone material. The flexible body 210 is configured to be deformable according to the shape of the user's head. Specifically, the flexible body 210 may have a curvature that is deformable according to the size of the user's forehead. The flexible body 210 may be formed to be curved in a direction contacting the user's body. The flexible body 210 may have bending grooves 212 formed on a surface contacting the user's body.

The bending grooves 212 can adjust the curvature of the flexible body 210. A plurality of the bending grooves 212 may be formed on one surface of the flexible body 210. For example, the bending grooves 212 may be provided as a pair and formed symmetrically on left and right sides of the flexible body 210. The bending grooves 212 may be formed between a region in which the first electrodes 224a are disposed and a region in which the second electrodes 224b are disposed.

The bending grooves 212 according to an embodiment of the present invention may have an open shape toward an upper side of the flexible body 210 and a closed shape toward a lower side thereof. Accordingly, when both ends of the flexible body 210 are pressed, the flexible body 210 bends at different curvatures due to a density difference between the upper side and the lower side, so that the upper side of the flexible body 210 has a greater curvature deformation rate than the lower side. That is, the region in which the first electrodes 224a are disposed and the region in which the second electrodes 224b are disposed may be formed at different inclination angles.

The electrode unit 220 has one side coupled to the body unit 100 and another side fixed to the flexible body 210. The electrode unit 220 is capable of contacting the user's body from the body unit 100 to detect at least one biosignal. The electrode unit 220 includes electrode bodies 222 and electrodes 224.

The electrode bodies 222 each have one side connected to the body unit 100 and another side in contact with the flexible body 210. The electrode bodies 222 are electrically connected to the body unit 100 and capable of transmitting electrical signals. Each electrode body 222 has a protrusion 2221 formed on one side thereof for coupling with the body unit 100. For example, the protrusion 2221 may have a circular T-bolt shape and may be connected to the insertion groove 1144 and the support tab groove 1244.

The electrode bodies 222 may be provided in a plurality. Hereinafter, it will be described that the electrode bodies 222 according to an embodiment of the present invention are composed of four. However, the number of the electrode bodies 222 provided in the biosignal measurement device 10 may be variously changed depending on the intended use and the position at which the device contacts the body. The electrode bodies 222 include first electrode bodies 222a and second electrode bodies 222b.

The first electrode bodies 222a are provided as a pair and coupled to the insertion grooves 1144 of the lower cover 114. The second electrode bodies 222b are provided as a pair and coupled to the support tab grooves 1244 of the support tabs 1242. Here, an opening of the insertion groove 1144 is blocked by the coupling of the upper cover 112 and the lower cover 114, and an opening of the support tab groove 1244 is formed with a narrower gap than an inner portion thereof to prevent detachment of the protrusion 2221. In addition, the protrusion 2221 of each electrode body 222 may move along the longitudinal direction of the insertion groove 1144 and the support tab groove 1244 when a position of the elastic pressing member 120 and a shape of the flexible body 210 are changed.

In addition, a distance between the pair of first electrode bodies 222a is formed to be narrower than a distance between the pair of second electrode bodies 222b. That is, the first electrode bodies 222a and the second electrode bodies 222b are disposed on one surface of the flexible body 210, with the first electrode bodies 222a disposed adjacent to a central region of the flexible body 210 and the second electrode bodies 222b disposed adjacent to both ends of the flexible body 210.

The electrodes 224 each have one side coupled to the electrode body 222 and another side in contact with the user's body. The electrodes 224 can detect EEG (Electroencephalography), ECG (Electrocardiography), and EMG (Electromyography) by sensing minute movements or vibrations of the body. The electrodes 224 may be provided in a plurality. The electrodes 224 may be provided in a number corresponding to the electrode bodies 222. For example, the plurality of electrodes 224 includes a pair of first electrodes 224a coupled to the pair of first electrode bodies 222a, and a pair of second electrodes 224b coupled to the second electrode bodies 222b. The electrode bodies 222 and the electrodes 224 are disposed on the flexible body 210 in front and rear directions and fixed to the flexible body 210. Here, the flexible body 210 has through-holes 214 formed therein to electrically connect the electrode bodies 222 and the electrodes 224.

The through-holes 214 may be formed in a number corresponding to the electrode bodies 222. The through-holes 214 may be formed to be smaller than the electrode bodies 222 and the electrodes 224 in size. The through-holes 214 may be formed closer to an upper side than a lower side of the flexible body 210.

In addition, the first electrodes 224a may be disposed so as to have an inclination angle different from that of the second electrodes 224b. For example, the first electrodes 224a are connected to the insertion grooves 1144 of the lower cover 114 through the first electrode bodies 222a so that the inclination angle thereof is not changed, whereas the second electrodes 224b are connected to the rotation brackets 124 through the second electrode bodies 222b so that the inclination angle and the tilting angle thereof may be changed according to a degree of deformation of the flexible body 210.

As described above, according to an embodiment of the present invention, when the plurality of electrodes 224 are in contact with the user's forehead, with the position of the first electrodes 224a being fixed according to the curvature of the forehead, an assembly composed of the second electrodes 224b, the flexible body 210, the second electrode bodies 222b, and the rotation brackets 124 is pushed in a direction opposite to the forehead, and the elastic body 126 presses the rotation brackets 124 toward the user's forehead, so that all of the electrodes 224 can be maintained in complete close contact with the user's body.

Although all the components constituting the embodiments of the present invention have been described as being combined into one or operating in combination, the present invention is not necessarily limited to such embodiments. That is, within the scope of the object of the present invention, all of the components may be selectively combined in one or more to operate. In addition, terms such as "include," "comprise," or "have" described above mean that the corresponding component may be present unless specifically stated to the contrary, and thus should be construed as being able to further include other components rather than excluding other components. All terms, including technical or scientific terms, have the same meaning as commonly understood by a person of ordinary skill in the art to which the present invention belongs unless otherwise defined. Terms commonly used, such as those defined in dictionaries, should be interpreted as consistent with their meanings in the context of the relevant technology and should not be interpreted in an idealized or overly formal sense unless explicitly defined in the present invention.

The foregoing description is merely illustrative of the technical idea of the present invention, and various modifications and variations can be made by those of ordinary skill in the art to which the present invention belongs without departing from the essential characteristics of the present invention. Therefore, the embodiments disclosed in the present invention are not intended to limit the technical idea of the present invention but to describe it, and the scope of the technical idea of the present invention is not limited by such embodiments. The scope of protection of the present invention should be construed by the claims below, and all technical ideas within an equivalent scope thereof should be construed as being included in the scope of rights of the present invention.

What is claimed is:

1. A biosignal measurement device comprising:
a body unit; and
a measurement unit disposed on one side of the body unit and configured to contact a user's body to detect biosignals,
wherein the body unit comprises:
a housing provided with a communication module and a controller; and
a pair of elastic pressing members, each having one side connected to the housing and another side connected to the measurement unit, the pair of elastic pressing members configured to press the measurement unit toward the user's body.

2. The biosignal measurement device of claim 1, wherein the elastic pressing member comprises:
a rotation shaft fixed to the housing;
a rotation bracket rotatably connected to the rotation shaft; and
an elastic body configured to press the rotation bracket toward the user's body.

3. The biosignal measurement device of claim 1, wherein the measurement unit comprises:
a flexible body having a curvature that is deformable along a contour of the user's body; and
a plurality of electrodes disposed on a surface of the flexible body that contacts the user's body.

4. The biosignal measurement device of claim 3, wherein the plurality of electrodes comprises:
a pair of first electrodes fixed to the housing; and
a pair of second electrodes each fixed to a respective one of the pair of elastic pressing members.

5. The biosignal measurement device of claim 4, wherein the flexible body is formed such that a region in which the first electrodes are disposed and a region in which the second electrodes are disposed have different inclination angles.

6. The biosignal measurement device of claim 5, wherein the first electrodes are disposed so as to have an inclination angle different from that of the second electrodes.

7. The biosignal measurement device of claim 4, wherein the second electrodes are configured such that a tilting angle thereof is changed by an elastic force of the elastic pressing members when worn by the user.

8. The biosignal measurement device of claim 1, wherein the body unit has a coupling portion formed on one surface thereof, the coupling portion configured to engage with a head-mounted display (HMD) device.
